(19) United States
(12) Patent Application Publication
(10) Pub. No.: US 2023/0012616 A1
(43) Pub. Date: Jan. 19, 2023

(54) BELT AND ELECTROCARDIOGRAPHIC MEASUREMENT DEVICE

(71) Applicant: OMRON HEALTHCARE Co., Ltd., Kyoto (JP)

(72) Inventors: Reiji FUJITA, Kyoto (JP); Akito ITO, Kyoto (JP); Yasuhiro KAWABATA, Kyoto (JP)

(21) Appl. No.: 17/934,920

(22) Filed: Sep. 23, 2022

Related U.S. Application Data
(63) Continuation of application No. PCT/JP2021/007617, filed on Mar. 1, 2021.

(30) Foreign Application Priority Data
Mar. 31, 2020 (JP) 2020-062874

(51) Int. Cl. A61B 5/256; A61B 5/282; A61B 5/332

(52) U.S. Cl. CPC A61B 5/256; A61B 5/282; A61B 5/332; A61B 5/339

(57) ABSTRACT

To provide a belt that can be closely attached to an upper arm, and an electrocardiographic measurement apparatus. A belt includes a belt body formed in a band shape, and an electrode including a plurality of electrode pieces disposed on a main surface of the belt body, located side by side in a short direction of the belt body, and connected in series by a signal line.

### CROSS-REFERENCE TO RELATED APPLICATIONS

This application is the U.S. national stage application filed pursuant to 35 U.S.C. 365(c) and 120 as a continuation of International Patent Application No. PCT/JP2021/007617, filed Mar. 1, 2021, which application claims priority to Japanese Patent Application No. 2020-062874, filed Mar. 31, 2020, which applications are incorporated herein by reference in their entireties.

### TECHNICAL FIELD

The present invention relates to a belt and an electrocardiographic measurement apparatus that are used in measurement of a biological signal in accordance with an electric potential generated due to cardiac motion on the surface of a living body.

### BACKGROUND ART

An electrocardiographic measurement apparatus is known that detects, as one of biological signals, an electrocardiographic signal, which is a voltage generated due to cardiac motion on the surface of a living body, and generates an electrocardiographic waveform of a user.

As such an electrocardiographic measurement apparatus, an electrocardiographic measurement apparatus is known that uses a belt including a belt body to be wound around the upper arm of a user, and a plurality of electrodes fixed to an inner surface of the belt body at equal intervals in a longitudinal direction (for example, see Patent Document 1).

### CITATION LIST—PATENT LITERATURE

Patent Document 1: JP 5428889 B

### SUMMARY OF INVENTION

### Technical Problem

In the aforementioned electrocardiographic measurement apparatus, the area of the electrode that comes into contact with the living body is preferably large in order to increase detected intensity of an electrocardiographic signal. Therefore, it is conceivable to increase the area of the surface of the electrode that comes into contact with the upper arm, and for example, it is conceivable to form the electrode into a large shape in the range from one end to the other end in a short direction of the belt.

However, the surface of the living body is configured into a curved surface. Therefore, when the area of the surface on the living body side of the electrode is increased, the region that does not come into contact with the living body increases. In other words, the degree of adhesion of the electrode to the living body decreases.

The present invention is thus intended to provide a belt that allows electrodes to be closely attached to a living body, and an electrocardiographic measurement apparatus.

### Solution to Problem

According to an aspect of the present invention, a belt includes a belt body formed in a band shape, and an electrode including a plurality of electrode pieces disposed on one main surface of the belt body, the plurality of electrode pieces located side by side in a short direction of the belt body, the plurality of electrode pieces connected in series by a signal line.

According to this aspect, the belt body is wound around a living body, and thus the plurality of electrode pieces are disposed along with the surface of the living body facing to the plurality of electrode pieces. In other words, the orientation of the plurality of electrode pieces varies along with the surface of the living body. Additionally, in other words, the surface on the living body side of the electrode, which is formed of the surface on the living body side of the plurality of electrode pieces is disposed along with the living body. Therefore, the electrode is closely attached to the living body.

In the belt of the aspect described above, the belt is provided in which lengths along the short direction of the plurality of electrode pieces are equal.

According to this aspect, the electrode can be formed of the electrode pieces having the same shape and the same size, and thus manufacturing costs of the electrode can be reduced.

In the belt of the aspect described above, the belt is provided in which the plurality of electrode pieces are three electrode pieces, and of the plurality of electrode pieces, electrode pieces disposed at both ends in the short direction each have a length along the short direction, which is short compared to a length along the short direction of an electrode piece, disposed in the middle in the short direction, of the three electrode pieces.

According to this aspect, both end sides of the electrode in the short direction of the belt body, which are difficult to closely attach to the living body, can be closely attached to the living body while the number of electrode pieces included in the electrode from increasing. Additionally, since the number of electrode pieces can be prevented from increasing, the number of signal lines can be reduced, and thus noise generated by the signal lines can be reduced.

In the belt of the aspect described above, the belt can be provided in which a plurality of the electrodes are disposed.

According to this aspect, the plurality of electrodes can be closely attached to the living body.

According to an aspect of the present invention, an electrocardiographic measurement apparatus is provided that includes the belt according to the aspect described above, a device body including a control board built in the device body, and a wiring portion disposed on the belt body and electrically connecting the control board and the electrode.

According to this aspect, the belt body is wound around the living body, and thus the plurality of electrode pieces are disposed along with the surface of the living body facing to the plurality of electrode pieces. In other words, the orientation of the plurality of electrode pieces varies along with the surface of the living body. Additionally, in other words, the surface on the living body side of the electrode, which is formed of the surface on the living body side of the plurality of electrode pieces is disposed along with the living body. Therefore, the electrode is closely attached to the living body. Since the electrode is closely attached to the living body, the electrocardiographic measurement apparatus can suitably detect the voltage generated on the surface of the living body and thus can more accurately measure an electrocardiographic waveform.

### Advantageous Effects of Invention

The present invention can provide a belt that allows electrodes to closely attach to a living body, and an electrocardiographic measurement apparatus.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is an explanatory diagram illustrating a state where an electrocardiographic measurement apparatus according to one embodiment of the present invention is attached to the upper arm of a user;

FIG. 2 is a block diagram illustrating the configuration of the electrocardiographic measurement apparatus;

FIG. 3 is a plan view illustrating the configuration of the electrocardiographic measurement apparatus;

FIG. 4 is a perspective view illustrating the configuration of the electrocardiographic measurement apparatus;

FIG. 5 is a cross-sectional view schematically illustrating a state where the electrocardiographic measurement apparatus is attached to the upper arm;

FIG. 6 is a plan view illustrating the configuration of the electrocardiographic measurement apparatus according to a modified example of one embodiment of the present invention;

FIG. 7 is a cross-sectional view schematically illustrating a state where the electrocardiographic measurement apparatus is attached to the upper arm;

FIG. 8 is a plan view illustrating the configuration of the electrocardiographic measurement apparatus according to a modified example of one embodiment of the present invention;

FIG. 9 is a cross-sectional view schematically illustrating a state where the electrocardiographic measurement apparatus is attached to the upper arm; and,

FIG. 10 is a plan view illustrating the configuration of the electrocardiographic measurement apparatus according to a modified example of one embodiment of the present invention.

### DESCRIPTION OF EMBODIMENTS

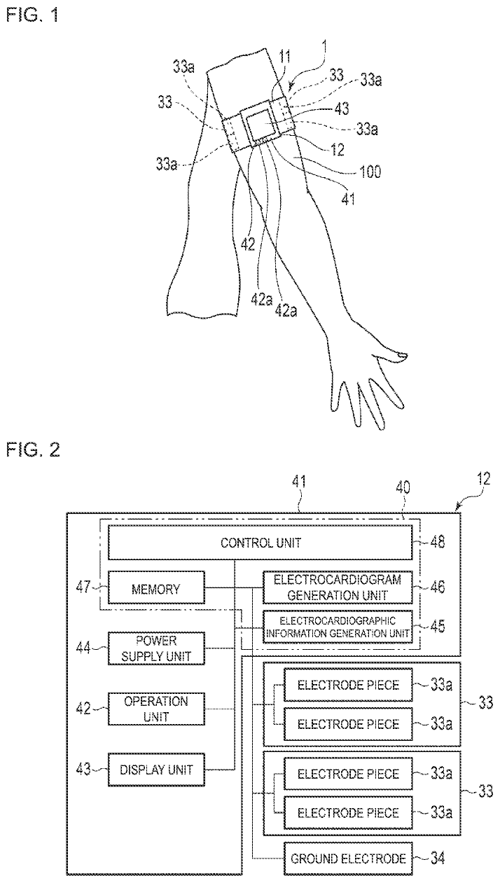

Hereinafter, an example of an electrocardiographic measurement apparatus 1 according to one embodiment of the present invention will be described with the use of FIG. 1 to FIG. 5.

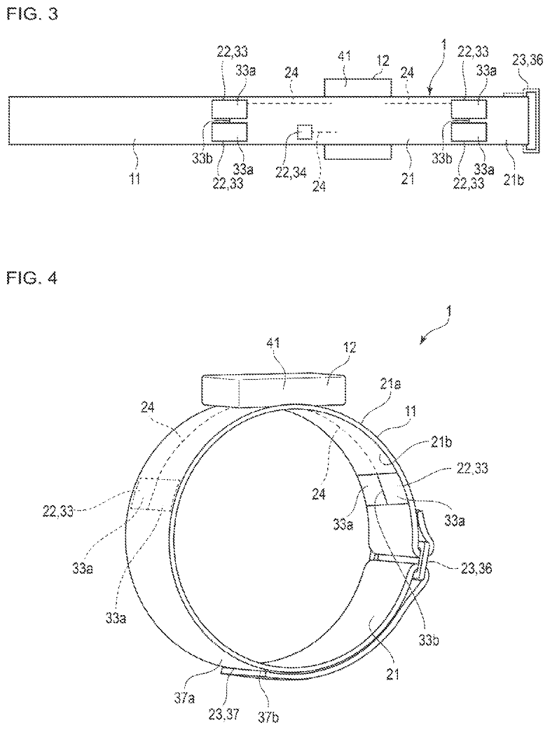

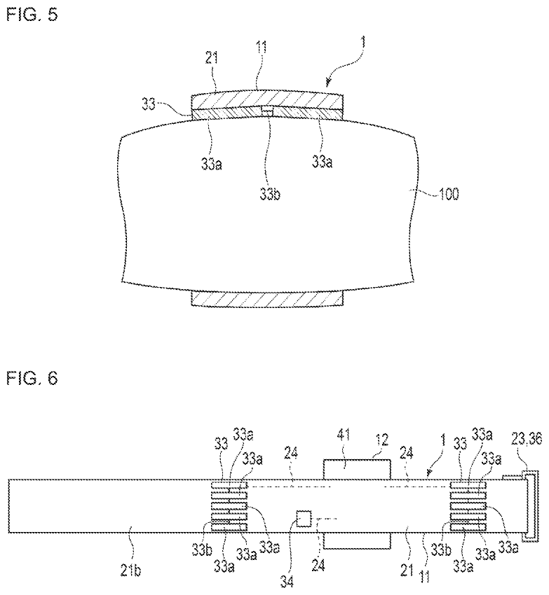

FIG. 1 is an explanatory diagram illustrating a state where the electrocardiographic measurement apparatus 1 is attached to an upper arm 100 of a user. FIG. 2 is a block diagram illustrating the configuration of the electrocardiographic measurement apparatus 1. FIG. 3 is a plan view illustrating the configuration of the electrocardiographic measurement apparatus 1. FIG. 3 illustrates a state where a belt 11 of the electrocardiographic measurement apparatus 1 is developed. FIG. 4 is a perspective view illustrating the configuration of the electrocardiographic measurement apparatus 1. FIG. 5 is a cross-sectional view schematically illustrating a state where the electrocardiographic measurement apparatus 1 is attached to the upper arm 100.

The electrocardiographic measurement apparatus 1 is an electric potential measurement apparatus that is attached to a living body to detect electric potentials at a plurality of points on the skin surface of the living body and generate electrocardiographic information necessary for generating an electrocardiogram based on the detected voltage. Note that the electrocardiographic measurement apparatus 1 may generate an electrocardiographic waveform and display the electrocardiographic waveform and may be configured to display information necessary for generating an electrocardiogram and output the information to an external terminal.

As illustrated in FIG. 1 to FIG. 3, the electrocardiographic measurement apparatus 1 includes the belt 11 and a device body 12. In the electrocardiographic measurement apparatus 1, for example, the belt 11 and the device body 12 are integrally formed. The electrocardiographic measurement apparatus 1 functions as, for example, a so-called wearable device to be attached by the belt 11 to the upper arm 100 as a living body. FIG. 1 illustrates an example of a state where the electrocardiographic measurement apparatus 1 is attached to the upper arm 100 of the subject. Note that the electrocardiographic measurement apparatus 1 may include the belt 11 and the device body 12 that are configured as separate components and are connected via a signal line or the like.

The belt 11 holds the device body 12. The belt 11 is wrapped around the upper arm 100. As illustrated in FIG. 3 and FIG. 4, the belt 11 includes a belt body 21, electrode arrays 22, fixing means 23, and a wiring portion 24.

The belt body 21 is formed of, for example, a resin or a fiber having flexibility. The belt body 21 is set to have a length that can be attached to the upper arm 100 of the subject wearing the electrocardiographic measurement apparatus 1. The belt body 21 is formed in a band shape that is long in one direction. The belt body 21 is configured such that when the electrocardiographic measurement apparatus 1 is attached to the upper arm 100, the device body 12 is fixed to the surface, which is the outer surface and the electrode arrays 22 are disposed on the back surface, which is the surface on the living body side.

The electrode arrays 22 are electrically connected via the wiring portion 24 to a control board 40 described below of the device body 12. The electrode arrays 22 include a plurality of electrodes 33 and a ground electrode 34.

The plurality of electrodes 33 are disposed on the other main surface 21b of the belt body 21. The plurality of electrodes 33 are disposed separated in a longitudinal direction of the belt body 21. The plurality of electrodes 33 are electrically connected via the wiring portion 24 to an electrocardiographic information generation unit 45 described below. In other words, the plurality of electrodes 33 are electrically connected to the control board 40. The plurality of electrodes 33 are, for example, two electrodes 33.

The electrodes 33 include a plurality of electrode pieces 33a that are disposed on the belt body 21 and located side by side in a short direction of the belt body 21 and are configured to vary the orientation along with the upper arm 100 when the belt body 21 is wrapped around the upper arm 100, and a signal line 33b that connects the plurality of electrode pieces 33a in series.

The plurality of electrode pieces 33a are formed of a conductive material. The plurality of electrode pieces 33a are fixed to the belt body 21. The plurality of electrode pieces 33a are formed, for example, in the same shape and in the same size. The plurality of electrode pieces 33a are each formed, for example, in a rectangular plate shape. A main surface on the upper arm side of the electrode piece 33a is, for example, configured in a flat surface.

The plurality of electrode pieces 33a are fixed to the belt body 21, for example, in the orientation such that two sides are in parallel in the short direction of the belt body 21. Additionally, the plurality of electrode pieces 33a are disposed such that two sides along the short direction of the belt body 21 are linearly arranged side by side. The length of the electrode piece 33a along the short direction of the belt body 21 is set to a length such that the sum of the lengths of the plurality of electrode pieces 33a along the short direction of the belt body 21 is a value as close as possible to the length along the short direction of the belt body 21.

A gap is disposed between the two electrode pieces 33a disposed adjacent to each other in the short direction of the belt body 21, the gap allowing the two electrode pieces 33a to be displaceable along with the upper arm 100.

The plurality of electrodes 33 configured as just described may be configured, for example, to be detachable and attachable with respect to the belt body 21.

The signal line 33b connects the plurality of electrode pieces 33a in series. In the present embodiment, the signal line 33b is connected to one electrode piece 33a and the other electrode piece 33a. The signal line 33b may be disposed, for example, inside the belt body 21.

The ground electrode 34 is disposed on the other main surface 21b of the belt body 21. The ground electrode 34 is electrically connected via the wiring portion 24 to the electrocardiographic information generation unit 45. In other words, the ground electrode 34 is electrically connected to the control board 40. The ground electrode 34 configured as just described may be configured, for example, to be detachable and attachable with respect to the belt body 21.

The wiring portion 24 electrically connects the plurality of electrodes 33 and the electrocardiographic information generation unit 45. Also, the wiring portion 24 electrically connects the ground electrode 34 and the electrocardiographic information generation unit 45.

The fixing means 23 is configured to fix the belt body 21 to the upper arm 100 in a state where the belt body 21 is wrapped around the upper arm 100 with the plurality of electrodes 33 in contact with the upper arm 100. The fixing means 23 includes, for example, a belt fixing ring 36 and a hook-and-loop fastener 37.

The belt fixing ring 36 is disposed at one end of the belt body 21 in the longitudinal direction. The belt fixing ring 36 is formed in an annular shape into which the belt body 21 can be inserted.

The hook-and-loop fastener 37 includes a loop 37a and a hook 37b. The loop 37a is disposed on one main surface 21a of the belt body 21. The loop 37a is disposed in a region of the main surface 21a, which faces the other end portion of the belt body 21, for example, folded back in the belt fixing ring 36.

Here, the region of the main surface 21a, which faces the other end portion of the belt body 21, is set to the size such that the region faces the other end portion of the belt body 21 even when the circumferential length of the upper arm 100 of the intended user is any circumferential length from the shortest length to the longest length. The hook 37b is disposed on the other end portion on the main surface 21a of the belt body 21.

The wiring portion 24 is formed, for example, of a lead wire embedded in the belt body 21. Also, the wiring portion 24 may be formed of a flexible substrate disposed on the main surface 21b of the belt body 21.

As illustrated in FIG. 1 to FIG. 3, the device body 12 includes a case 41, an operation unit 42, a display unit 43, a power supply unit 44, the electrocardiographic information generation unit 45, an electrocardiogram generation unit 46, a memory 47, and a control unit 48.

The electrocardiographic information generation unit 45, the electrocardiogram generation unit 46, and the control unit 48 are disposed, for example, on the control board 40. The control board 40 is housed in the case 41. Additionally, the operation unit 42 and the display unit 43 are disposed, for example, on the case 41. Further, the device body 12 includes a communication unit that transmits and receives information with an external terminal. Note that the communication unit transmits and receives information with an external terminal wirelessly and/or in a wired manner. The communication unit is disposed, for example, on the control board 40.

The case 41 houses a portion of the operation unit 42, a portion of the display unit 43, the electrocardiographic information generation unit 45, the electrocardiogram generation unit 46, the memory 47, and the control unit 48. Additionally, the case 41 exposes a portion of the operation unit 42 and a portion of the display unit 43 from the outer surface. The case 41 is fixed to the belt 11.

The operation unit 42 is configured to receive an instruction input from a user. For example, the operation unit 42 includes a plurality of buttons 42a and a sensor that detects operations of the buttons 42a. Note that the operation unit 42 may include a touch panel of a pressure sensitive type, a capacitance type, or the like, a microphone that receives an instruction by sound, or the like, which are disposed on the case 41, the display unit 43, or the like. When operated by the user, the operation unit 42 converts the instruction into an electrical signal and outputs the electrical signal to the control unit 48.

The display unit 43 is electrically connected to the control unit 48. The display unit 43 is, for example, a liquid crystal display (LCD) or an organic electro luminescence display (OELD). The display unit 43 displays date and time, electrocardiographic information, an electrocardiographic waveform, or the like in accordance with a control signal from the control unit 48. Note that, in a case where the electrocardiographic measurement apparatus 1 is used in a biological information measurement device that displays a blood pressure value, the display unit 43 may display a variety of information including measurement results of blood pressure values such as systolic blood pressure and diastolic blood pressure, a heart rate, or the like.

The power supply unit 44 is a power source. The power supply unit 44 is, for example, a rechargeable battery such as a lithium ion battery. The power supply unit 44 is electrically connected to the control unit 48. As a specific example, the power supply unit 44 supplies power to the control unit 48. The power supply unit 44 supplies power for drive to the control unit 48, and via the control unit 48 to the operation unit 42, the display unit 43, the electrocardiographic information generation unit 45, the electrocardiogram generation unit 46, and the memory 47.

The electrocardiographic information generation unit 45 is electrically connected, for example, via the wiring portion 24 to the plurality of electrodes 33 of the electrode arrays 22, to two electrodes 33 in the present embodiment. The electrocardiographic information generation unit 45 calculates a potential difference from voltages detected by the two electrodes 33 and generates electrocardiographic information.

The electrocardiogram generation unit 46 is electrically connected to the electrocardiographic information generation unit 45. The electrocardiogram generation unit 46 generates information of an electrocardiogram based on the electrocardiographic information generated by the electrocardiographic information generation unit 45. The information of the electrocardiogram may include electrocardiographic waveforms.

The electrocardiographic information generation unit 45 and the electrocardiogram generation unit 46 as just described are processing circuits that can respectively execute, for example, functions of the electrocardiographic information generation unit 45 and the electrocardiogram generation unit 46. The electrocardiographic information generation unit 45 and the electrocardiogram generation unit 46 are electrically connected to the control unit 48. Note that the control unit 48 includes the processing circuits of the electrocardiographic information generation unit 45 and the electrocardiogram generation unit 46 and executes a program stored in the memory 47 and thus may perform the functions of the electrocardiographic information generation unit 45 and the electrocardiogram generation unit 46.

Also, for example, the electrocardiographic information generation unit 45 or the electrocardiogram generation unit 46 may include a low pass filter, an amplifier, and an analog/digital converter. For example, unnecessary noise components are removed from a signal of the potential difference by the low pass filter, and the signal is amplified by the amplifier and then converted into a digital signal by the analog/digital converter.

The memory 47 includes a solid state drive (SSD), a random access memory (RAM), a read only memory (ROM), and the like. The memory 47 stores programs necessary for executing a variety of control processing. Further, the memory 47 stores the detected electrocardiographic signal, the generated electrocardiographic information and electrocardiogram information, and the like. Furthermore, for example, the memory 47 stores these pieces of information in a chronological order.

The control unit 48 includes one or a plurality of processors. The control unit 48 is formed of one or more processing circuits. The control unit 48 is, for example, a central processing unit (CPU). The control unit 48 causes the entire operation and predetermined operation (function) of the electrocardiographic measurement apparatus 1 to be executed based on the programs stored in the memory 47. The control unit 48 executes the predetermined operation, analysis, processing, or the like according to the read program. The control unit 48 controls operation of the operation unit 42, the display unit 43, the electrocardiographic information generation unit 45, and the electrocardiogram generation unit 46, transmits/receives a signal, and supplies power.

An example of attachment of the electrocardiographic measurement apparatus 1 configured as just described to the upper arm 100 will be described. For example, the subject wraps the belt body 21 around the upper arm 100 with the main surface 21b of the belt body 21 facing to the upper arm 100 of the subject, and inserts the other end portion of the belt body 21 in the belt fixing ring 36 to fold back the other end portion in the belt fixing ring 36. Next, the subject pulls the other end portion of the belt body 21 and thereby tightens the belt body 21 to the upper arm 100. Then, the subject fixes the hook 37b disposed on the other end portion of the belt body 21 to the loop 37a disposed on the belt body 21.

Therefore, the electrocardiographic measurement apparatus 1 is attached to the upper arm of the subject. Then, the subject operates the operation unit 42, and thus the control unit 48 controls each configuration and detects an electrocardiographic signal via the two electrodes 33. Then, the electrocardiographic information generation unit 45 generates electrocardiographic information from the electrocardiographic signal, and the electrocardiogram generation unit 46 generates electrocardiogram information from the electrocardiographic information. The control unit 48 allows the memory 47 to store the electrocardiographic information and the electrocardiogram information and allows the display unit 43 to display information such as a date and time and an electrocardiogram. Also, the control unit 48 may control the communication unit to transmit a variety of information such as the date and time, the electrocardiographic information, and the electrocardiogram information to an external terminal.

In the electrocardiographic measurement apparatus 1 configured as just described, the belt body 21 is wrapped around the upper arm 100, and thus the plurality of electrode pieces 33a of each of the plurality of electrodes 33 vary the orientation along with a portion of the upper arm 100 in which the plurality of electrode pieces 33a are disposed. In other words, the surface on the upper arm 100 side of the electrodes 33, which is configured by the surface on the upper arm 100 side of the plurality of electrode pieces 33a varies along with the upper arm 100. Therefore, the electrodes 33 are closely attached to the upper arm 100. Since the electrodes 33 are closely attached to the upper arm 100, the electrocardiographic measurement apparatus 1 can suitably detect the voltage generated on the surface of the upper arm 100. As a result, the electrocardiographic measurement apparatus 1 can more accurately measure an electrocardiogram waveform.

Further, the plurality of electrode pieces 33a are formed in the same shape and in the same size. Therefore, manufacturing costs of the electrodes 33 can be reduced.

Furthermore, each of the plurality of electrodes 33 includes the plurality of electrode pieces 33a. Therefore, the plurality of electrodes 33 can be closely attached to the upper arm 100.

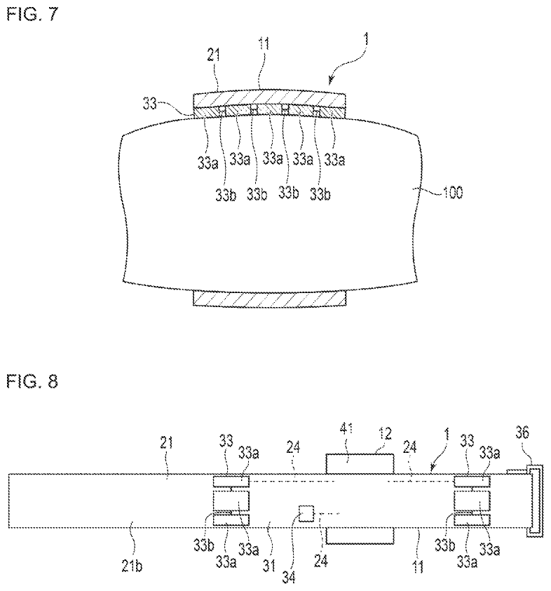

Note that in the aforementioned example, the configuration where the electrode 33 includes the plurality of electrode pieces 33a, for example, includes two electrode pieces 33a is described, but is not limited thereto. In other examples, the electrode 33 may be configured to include three or more electrode pieces 33a as in a modified example illustrated in FIG. 6 and FIG. 7. FIG. 6 is a plan view illustrating, as an example, the configuration of the electrocardiographic measurement apparatus 1 in which the electrode 33 includes five electrode pieces 33a. FIG. 7 is a cross-sectional view schematically illustrating a state where the electrocardiographic measurement apparatus 1 is attached to the upper arm 100 of a user.

Also, in the aforementioned example, the configuration where the plurality of electrode pieces 33a of the electrode 33 are formed in the same size is described as an example, but is not limited thereto. In other examples, the plurality of electrode pieces 33a may be formed such that at least one is formed in a different shape and in a different size as compared to the others.

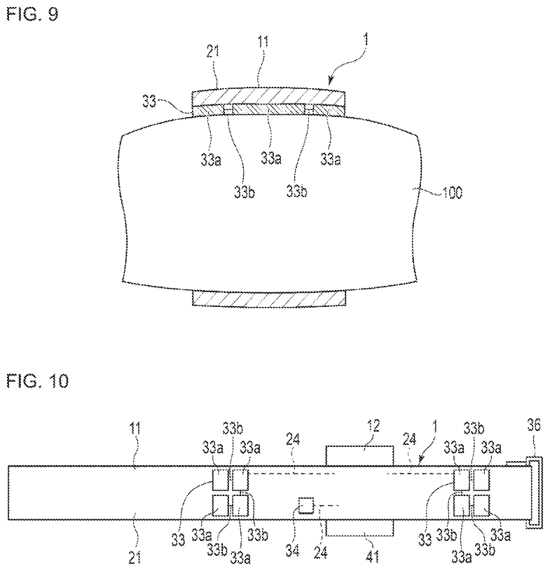

An example of the configuration where at least one of the plurality of electrode pieces 33a is formed in a different size as compared to the others is described with the use of FIG. 8 and FIG. 9. In a case where the electrode 33 includes three electrode pieces 33a as illustrated in FIG. 8 and FIG. 9, the length along the short direction of the belt body 21 of each of the two electrode pieces 33a disposed at both ends in the short direction of the belt body 21 is short compared to the length along the short direction of the belt body 21 of one of the three electrode pieces 33a, which is disposed in the middle in the short direction of the belt body 21.

As just described, of the three electrode pieces 33a, the length along the short direction of the belt body 21 of each of the two electrode pieces 33a disposed at the both ends in the short direction of the belt body 21 is short compared to the length along the short direction of the belt body 21 of the electrode piece 33a disposed in the middle in the short direction of the belt body 21. Therefore, the electrode 33 can be closely attached to the upper arm 100 while the number of electrode pieces 33a is prevented from increasing.

This effect will be described. In a case where the electrode 33 is formed of one member, the both end sides of the electrode 33 in the short direction of the belt body 21 are separated from the upper arm 100. However, the both end sides of the electrode 33 in the short direction are respectively formed of the electrode pieces 33a, and thus the electrode 33 can be closely attached to the upper arm 100.

Additionally, since the number of electrode pieces 33a can be prevented from increasing, and thus the number of signal lines 33b can be prevented from increasing. Therefore, noise detected by the electrode 33 can be reduced.

Moreover, in the aforementioned example, the configuration where each of the plurality of electrodes 33 includes the plurality of electrode pieces 33a is described as an example, but is not limited thereto. Each of one or more of the plurality of electrodes 33 may be configured to include the plurality of electrode pieces 33a.

For example, in the configuration where three electrodes 33 are provided as the plurality of electrodes 33, one of the three electrodes 33 may be formed of one member.

Further, in the aforementioned example, the configuration where the plurality of electrode pieces 33a of the electrode 33 are disposed side by side in a line in the short direction of the belt body 21 is described as an example, but is not limited thereto. In other examples, as illustrated in FIG. 10, the plurality of electrode pieces 33a may be disposed side by side in a plurality of lines in the short direction of the belt body 21. FIG. 10 illustrates, as an example, the electrocardiographic measurement apparatus 1 including the configuration where the electrode 33 includes four electrode pieces 33a and the four electrode pieces 33a are disposed such that two electrode pieces and two electrode pieces are located in two lines in the short direction of the belt body 21. In addition, the four electrode pieces 33a are connected in series by the signal line 33b.

Furthermore, in the aforementioned example, the configuration where the two electrodes 33 are disposed is described as an example, but is not limited thereto. In other examples, three or more electrodes 33 may be disposed.

Additionally, in the aforementioned example, the electrocardiographic measurement apparatus 1 is described with the use of an example in which the belt 11 is attached to the upper arm, but may be configured to be attached to the chest or to other portions of the living body.

Additionally, in the aforementioned example, the configuration where the belt 11 is used in the electrocardiographic measurement apparatus 1 is described, but is not limited thereto. For example, the belt 11 may be used in a biological information measurement device used for electrocardiographic measurement and blood pressure measurement. As a specific example, in addition to the configuration of the electrocardiographic measurement apparatus 1 described above, the biological information measurement device may include a processing circuit or the like that exerts a function of blood pressure measurement, which generates a blood pressure value from a pulse wave sensor and pulse wave information detected by the pulse wave sensor. Such a biological information measurement device calculates a pulse transit time (PTT) per heartbeat, and performs a function of blood pressure measurement that measures the blood pressure value by estimating blood pressure. Note that such a biological information measurement device calculates the pulse transit time (PTT) per heartbeat based on, for example, a time difference between an R-wave peak RP detected by an electrocardiographic signal and a pulse wave rise PS per heartbeat, which is one of the feature values of a pulse wave signal detected by the pulse wave sensor.

While the embodiments according to the present invention have been described in detail above, the above-described description merely exemplifies the present invention in all respects, and obviously, various improvements and modifications can be made without departing from the scope of the present invention. That is, specific configurations according to the respective embodiment may be employed as appropriate in the implementation of the present invention.

Additionally, in the present invention, various inventions can be formed by appropriately combining a plurality of components disclosed in the embodiments described above. For example, some components may be omitted from all the components described in the respective embodiments. Further, the components of the different embodiments may be combined appropriately.

### REFERENCE NUMERALS LIST

1 Electrocardiographic measurement apparatus
11 Belt
12 Device body
21 Belt body
21a Main surface
21b Main surface
22 Electrode array
23 Fixing means
24 Wiring portion
33 Electrode
33a Electrode piece
33b Signal line
34 Ground electrode
36 Belt fixing ring
37 Hook-and-loop fastener
37a Loop
37b Hook
41 Case
42 Operation unit
42a Button
43 Display unit
44 Power supply unit
45 Electrocardiographic information generation unit
46 Electrocardiogram generation unit
47 Memory
48 Control unit
100 Upper arm

What is claimed is:

1. A belt, comprising:
a belt body formed in a band shape; and
an electrode including a plurality of electrode pieces disposed on one main surface of the belt body, the plurality of electrode pieces located side by side in a short direction of the belt body, the plurality of electrode pieces connected in series by a signal line.

2. The belt according to claim 1, wherein lengths along the short direction of the plurality of electrode pieces are equal.

3. The belt according to claim 1, wherein
the plurality of electrode pieces are three electrode pieces, and
of the plurality of electrode pieces, electrode pieces disposed at both ends in the short direction each have a length along the short direction, which is short compared to a length along the short direction of an electrode piece, disposed in the middle in the short direction, of the three electrode pieces.

4. The belt according to claim 1, wherein a plurality of the electrodes are disposed.

5. An electrocardiographic measurement apparatus comprising:
the belt according to claim 1;
a device body including a control board built in the device body; and
a wiring portion disposed on the belt body and electrically connecting the control board and the electrode.

6. An electrocardiographic measurement apparatus comprising:
the belt according to claim 2;
a device body including a control board built in the device body; and
a wiring portion disposed on the belt body and electrically connecting the control board and the electrode.

7. An electrocardiographic measurement apparatus comprising:
the belt according to claim 3;
a device body including a control board built in the device body; and
a wiring portion disposed on the belt body and electrically connecting the control board and the electrode.

8. An electrocardiographic measurement apparatus comprising:
the belt according to claim 4;
a device body including a control board built in the device body; and
a wiring portion disposed on the belt body and electrically connecting the control board and the electrode.
